(19) United States
(12) Patent Application Publication
(10) Pub. No.: US 2016/0015424 A1
(43) Pub. Date: Jan. 21, 2016

(54) APPARATUS AND METHOD FOR LOADING FOLLICLES

(71) Applicant: Electronics and Telecommunications Research Institute, Daejeon (KR)

(72) Inventors: Kyuhyung KIM, Daegu (KR); Tae Wuk BAE, Daegu (KR); Hyung Soo LEE, Daegu (KR); Chang Hyuk HONG, Daegu (KR); Soo In LEE, Daejeon (KR); Yong Chul JUNG, Daegu (KR)

(21) Appl. No.: 14/684,271

(22) Filed: Apr. 10, 2015

(30) Foreign Application Priority Data
Jul. 18, 2014 (KR) 10-2014-0091276

(51) Int. Cl. A61B 17/34; A61F 2/10

(52) U.S. Cl. CPC A61B 17/3468; A61F 2/10; A61B 2017/00752

(57) ABSTRACT

A follicle loading apparatus that is positioned in a follicle supplier and loads a follicle on an inlet of the follicle loading apparatus according to an air pressure, and then the inlet is moved into the follicle supplier so as to accommodate the follicle in the follicle supplier, is provided.

### CROSS-REFERENCE TO RELATED APPLICATION

This application claims priority to and the benefit of Korean Patent Application No. 10-2014-0091276 filed in the Korean Intellectual Property Office on Jul. 18, 2014, the entire contents of which are incorporated herein by reference.

### BACKGROUND OF THE INVENTION

(a) Field of the Invention

The present invention relates to an apparatus and method for loading follicles. More particularly, the present invention relates to an apparatus and method for loading follicles on a follicle supplier.

(b) Description of the Related Art

Currently, for hair transplantation, follicles are taken from a scalp of a patient who wants hair transplantation and then are isolated by a follicle isolator. Then a transplantation operator loads the follicles on a hair transplanter (i.e., a follicle supplier) one by one and transplants the follicle on the scalp of the patient using the hair transplanter with the follicles loaded thereon.

However, an operation of separately loading follicles on the hair transplanter is simplified and it is also difficult to load follicles with a size of about 1 mm on the hair transplanter using tweezers, etc., one by one.

The above information disclosed in this Background section is only for enhancement of understanding of the background of the invention and therefore it may contain information that does not form the prior art that is already known in this country to a person of ordinary skill in the art.

### SUMMARY OF THE INVENTION

The present invention has been made in an effort to provide an apparatus and method for loading follicles having advantages of rapidly loading follicles on a hair transplanter.

An exemplary embodiment of the present invention provides a follicle loading apparatus for supplying taken follicles to a follicle supplier. The follicle loading apparatus may include a body and an inlet. The body may have a hollow inner part and be disposed in the follicle supplier. In addition, the inlet may be formed at one end portion of the body and on which follicles are loaded according to an air pressure in the body.

The follicle loading apparatus may further include a pneumatic device coupled to the other end portion of the body and for generating air pressure in the body.

The body may accommodate the follicle loaded in the inlet in the follicle supplier while moving in the follicle supplier.

The follicle may be accommodated in the follicle supplier via the adjustment of an air pressure.

The air pressure may be stopped when the follicle is positioned at an appropriate position in the follicle supplier.

A length of the body may be formed to be longer than a length of the follicle supplier.

Another exemplary embodiment of the present invention provides a follicle loading method of a follicle loading apparatus for supplying taken follicles to a follicle supplier.

The follicle loading method may include generating an air pressure, moving an inlet of the follicle loading apparatus into the follicle supplier when the follicle is loaded on the inlet of the follicle loading apparatus according to the air pressure, and accommodating the follicle in the follicle supplier.

The accommodating may include stopping the air pressure from being supplied.

The moving may include moving the follicle loading apparatus in a length direction of the follicle supplier in the follicle supplier.

The moving in the length direction of the follicle supplier may include moving an inlet of the follicle loading apparatus to be close to the follicle, and moving the inlet of the follicle loading apparatus into the follicle supplier when the follicle is loaded on the inlet.

### BRIEF DESCRIPTION OF THE DRAWINGS

FIG. 1 is a drawing illustrating an example of a follicle loading apparatus according to a first exemplary embodiment of the present invention.

FIG. 2 is a drawing illustrating an example of a sectional view of a follicle supplier illustrated in FIG. 1.

FIG. 3 is a drawing illustrating a follicle loading apparatus according to a second exemplary embodiment of the present invention.

FIG. 4 is a drawing of an operating method of a follicle loading apparatus according to a first exemplary embodiment of the present invention.

FIG. 5 is a drawing illustrating follicle loading in association with a hair transplantation process according to an exemplary embodiment of the present invention.

FIG. 6 is a drawing illustrating an example of arrangement of follicle suppliers according to an exemplary embodiment of the present invention.

### DETAILED DESCRIPTION OF THE EMBODIMENTS

In the following detailed description, only certain exemplary embodiments of the present invention have been shown and described, simply by way of illustration. As those skilled in the art would realize, the described embodiments may be modified in various different ways, all without departing from the spirit or scope of the present invention. Accordingly, to clearly describe the present invention, parts without concern to the description are omitted in the drawings, and like reference numerals in the specification denote like elements.

Throughout the specification and claims, unless explicitly described to the contrary, the word “comprise” and variations such as “comprises” or “comprising” will be understood to imply the inclusion of other elements but not the exclusion of any other elements.

Hereinafter, a method and apparatus for loading follicles according to an exemplary embodiment of the present invention will be described in detail with reference to the drawings.

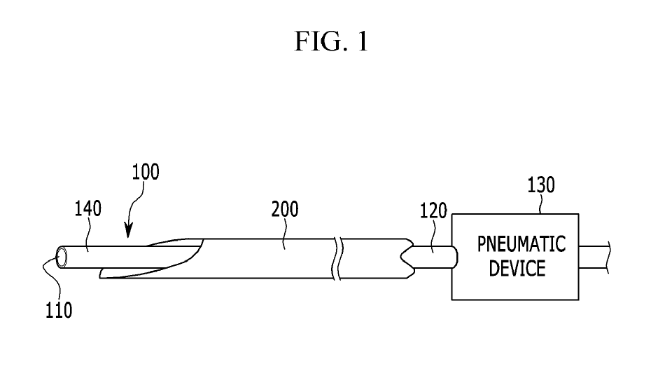

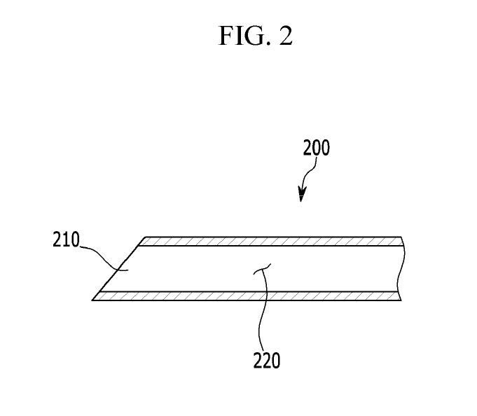

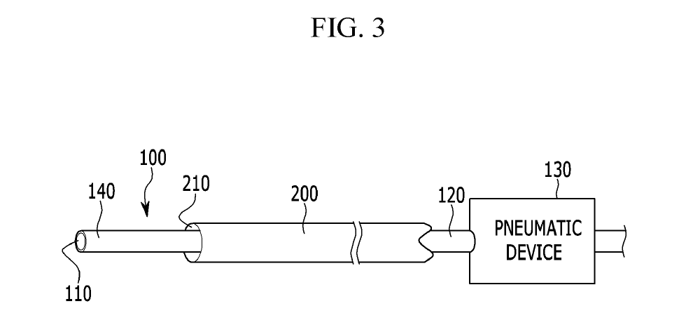

FIG. 1 is a drawing illustrating an example of a follicle loading apparatus 100 according to a first exemplary embodiment of the present invention, and FIG. 2 is a drawing illustrating an example of a sectional view of a follicle supplier 200 illustrated in FIG. 1. In addition, FIG. 3 is a drawing illustrating a follicle loading apparatus 100 according to a second exemplary embodiment of the present invention.

Referring to FIG. 1, the follicle loading apparatus 100 is used to load follicles on the follicle supplier 200, it moves in a length direction of the follicle supplier 200 in the follicle supplier 200, and it is formed to be longer than the follicle supplier 200 so as to move in the follicle supplier 200.

The follicle loading apparatus 100 includes a body 140 having a hollow inner part through which air passes, and sucks follicles using air pressure and loads the follicles on an inlet 110 of the follicle loading apparatus 100.

An end portion 120 of the body 140 positioned at an opposite side of the inlet 110 of the follicle loading apparatus 100 is coupled to a pneumatic device 130 for generating an air pressure. The pneumatic device 130 may receive supplied power to operate, and generate an air pressure in the follicle loading apparatus 100 and suck follicles toward the inlet 110 of the follicle loading apparatus 100 during the operation.

The size of the inlet 110 may be formed to be smaller than the size of the follicle so as to load the follicle on the inlet 110 without allowing the follicle into the follicle loading apparatus 100 through the inlet 110.

When the follicle is loaded on the inlet 110, the body 140 of the follicle loading apparatus 100 is moved backward in the follicle supplier 200 so as to position the follicle in the follicle supplier 200.

That is, the follicle loading apparatus 100 may be moved backward and forward according to user's instruction. That is, when the user instructs to move the follicle loading apparatus 100 forward, the follicle loading apparatus 100 is moved forward in the follicle supplier 200 according to the instruction for forward movement such that the inlet 110 of the follicle loading apparatus 100 is positioned close to the follicle. In addition, when the follicle is loaded on the inlet 110 of the follicle loading apparatus 100, the follicle loading apparatus 100 may be moved backward in the follicle supplier 200 according to user's instruction for backward movement. The follicle is positioned in the follicle supplier 200 via control of an air pressure. In this case, the instruction for backward and forward movement may be performed via a predetermined button.

When the follicle is positioned in the follicle supplier 200, the follicle loading apparatus 100 may be separated from the follicle supplier 200.

The follicle supplier 200 includes a movement space portion 220 having a hollow inner part. The follicle loading apparatus 100 is moved into the movement space portion 220 in a length direction and the moved follicle is positioned in the movement space portion 220 through an entry 210.

The follicle supplier 200 may be used for follicle transplantation in association with an automatic hair transplantation apparatus, and may be a component included in a follicle storage cartridge. That is, the follicle supplier 200 may be inserted into the follicle storage cartridge.

The follicle supplier 200 may be a needle used for hair transplantation or a device for supplying a follicle to the needle.

The follicle supplier 200 may have a needle type entry 210 with a sharp shape illustrated in FIG. 1, or a circular type entry 210 with a circular shape illustrated in FIG. 3.

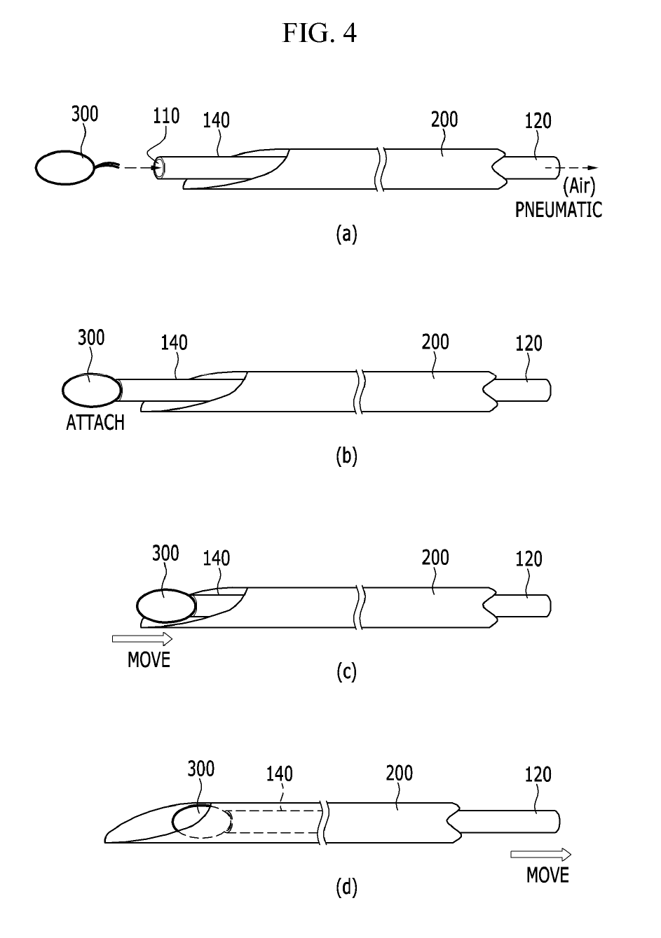

FIG. 4 is a drawing of an operating method of a follicle loading apparatus 100 according to an exemplary embodiment of the present invention.

Referring to FIG. 4(a), air pressure (pneumatic) is generated by a pneumatic device in a direction toward the end portion 120 in the inlet 110. In this case, when the inlet 110 of the follicle loading apparatus 100 is positioned close to a follicle 300, the follicle 300 is attached to the inlet 110 of the follicle loading apparatus 100 according to an air pressure.

When the follicle 300 is attached to the inlet 110 of the follicle loading apparatus 100, the follicle loading apparatus 100 is moved backward in the follicle supplier 200 so as to insert the inlet 110 of the follicle loading apparatus 100 into the follicle supplier 200, as illustrated in FIGS. 4(b) through 4(d).

Thus, the follicle 300 is accommodated in the follicle supplier 200. In this case, air pressure may be controlled to dispose the follicle 300 at an appropriate position in the follicle supplier 200. For example, when the follicle 300 is moved into the follicle supplier 200, intensity of air pressure is weakened to adjust movement speed of the follicle 300 so as to slowly move the follicle 300 in the follicle supplier 200, and when the follicle 300 is positioned at an appropriate position in the follicle supplier 200, the supply of the air pressure is stopped to accommodate the follicle 300 at the position.

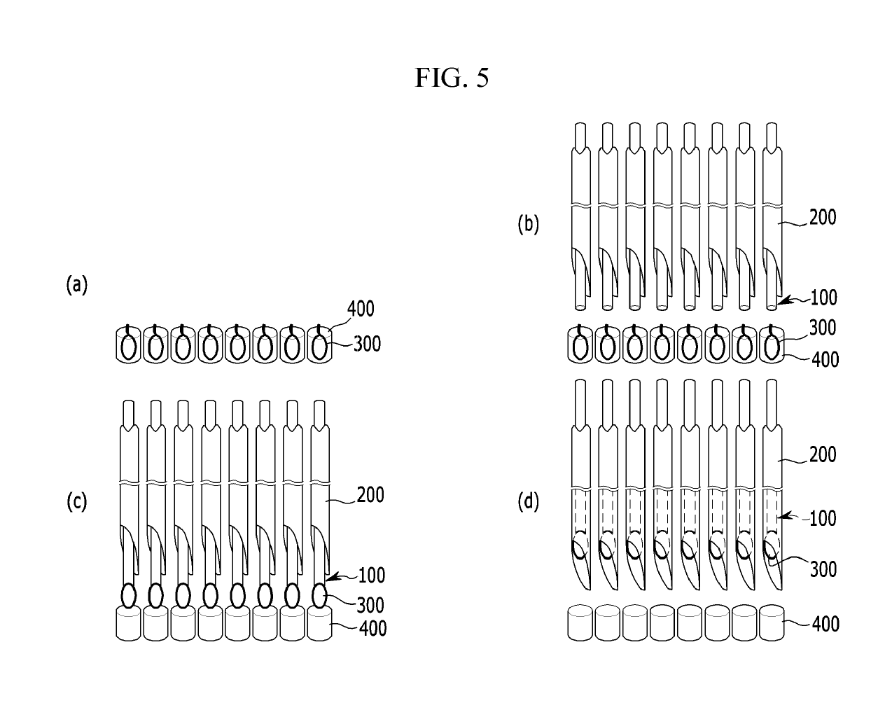

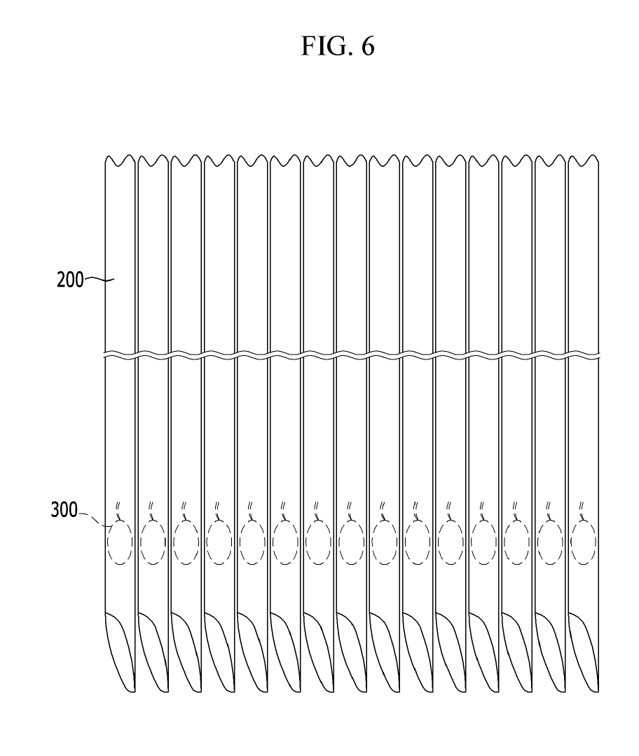

FIG. 5 is a drawing illustrating follicle loading in association with a hair transplantation process according to an exemplary embodiment of the present invention, and FIG. 6 is a drawing illustrating an example of arrangement of follicle suppliers according to an exemplary embodiment of the present invention.

As illustrated in FIG. 5(a), when each follicle 300 is stored in each storage 400, follicles may be sucked using a plurality of the follicle loading apparatuses 100 and accommodated in a plurality of the follicle suppliers 200, respectively, as illustrated in FIGS. 5(b) through 5(d).

In addition, a plurality of the follicle suppliers 200 may be arranged in a straight line illustrated in FIG. 6, and follicles 300 may be loaded in the follicle suppliers 200, respectively, and then an automatic hair transplantation apparatus may be used. Alternatively, the plurality of the follicle suppliers 200 may be arranged in a circular shape.

When the follicle suppliers 200 arranged in the straight line or circular shape are used, it is possible to insert the follicle suppliers 200 directly into a straight or circular type of cartridge.

According to an exemplary embodiment of the present invention, follicles may be rapidly and effectively loaded on a hair transplanter compared with a case in which a transplantation operator manually loads follicles on the hair transplanter, thereby overcoming difficulty in manually loading follicles.

The exemplary embodiments of the present invention may not be embodied via only the aforementioned apparatus and/or method, and may be embodied via a program for realizing a function corresponding to a configuration according to the exemplary embodiments of the present invention or a recording medium with the program recorded thereon. This embodiment may be easily realized by one of ordinary skill in the art to which the present pertains from the description of the aforementioned embodiments of the present invention.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

What is claimed is:

1. A follicle loading apparatus for supplying a taken follicle to a follicle supplier, comprising:
a body having a hollow inner part and disposed in the follicle supplier; and
an inlet formed at one end portion of the body and on which the follicle is loaded according to an air pressure in the body.

2. The follicle loading apparatus of claim 1, further comprising a pneumatic device coupled to the other end portion of the body and for generating air pressure in the body.

3. The follicle loading apparatus of claim 1, wherein the body accommodates the follicle loaded in the inlet in the follicle supplier while moving in the follicle supplier.

4. The follicle loading apparatus of claim 3, wherein the follicle is accommodated in the follicle supplier via adjustment of an air pressure.

5. The follicle loading apparatus of claim 3, wherein the air pressure is stopped when the follicle is positioned at an appropriate position in the follicle supplier.

6. The follicle loading apparatus of claim 1, wherein a length of the body is formed to be longer than a length of the follicle supplier.

7. A follicle loading method of a follicle loading apparatus for supplying a taken follicle to a follicle supplier, comprising:
generating an air pressure;
moving an inlet of the follicle loading apparatus into the follicle supplier when the follicle is loaded on the inlet of the follicle loading apparatus according to the air pressure; and
accommodating the follicle in the follicle supplier.

8. The follicle loading method according to claim 7, wherein the accommodating includes stopping the air pressure from being supplied.

9. The follicle loading method according to claim 7, wherein the moving includes moving the follicle loading apparatus in a length direction of the follicle supplier in the follicle supplier.

10. The follicle loading method according to claim 9, wherein the moving in the length direction of the follicle supplier includes:
moving an inlet of the follicle loading apparatus to be close to the follicle; and
moving the inlet of the follicle loading apparatus into the follicle supplier when the follicle is loaded on the inlet.
